(19) United States
(12) Patent Application Publication
(10) Pub. No.: US 2006/0222617 A1
(43) Pub. Date: Oct. 5, 2006

(54) Water-soluble cosmetic composition and method for manufacturing the same

(75) Inventors: Po-Han Chen, Tainan City (TW); Ya-Ping Hung, Tainan City (TW); De-Ching Chou, Tainan City (TW); Ying-Fei Huang, Tainan City (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: Taiyen Biotech. Co. Ltd.

(21) Appl. No.: 11/291,412

(22) Filed: Dec. 1, 2005

(30) Foreign Application Priority Data
Apr. 1, 2005 (TW) 94110632
Jun. 29, 2005 (TW) 94121967

(51) Int. Cl. A61K 8/64

(52) U.S. Cl. 424/70.14

(57) ABSTRACT

A water-soluble cosmetic composition and the manufacturing method thereof are provided. The water-soluble cosmetic composition is obtained by a process as follows. First, a mixed solution is provided, wherein the mixed solution comprises about 0.1% to 30% by weight of ascorbic acid or derivatives thereof, and 0.1% to 10% water-soluble collagen having molecular weight in the range of about 3 kD to 300 kD. Subsequently a vacuum dehydration process is conducted to remove a portion of water from the mixed solution.

### RELATED APPLICATIONS

The present application is based on, and claims priorities from, Taiwan Application Serial Number 94110632, filed Apr. 1, 2005, and Taiwan Application Serial Number 94121967, filed Jun. 29, 2005, the disclosure of which is hereby incorporated by reference herein in its entirety.

### FIELD OF THE INVENTION

The present invention relates to a water-soluble cosmetic composition, and more particularly, relates to a water-soluble cosmetic base with collagen and ascorbic acid or derivatives thereof.

### BACKGROUND OF THE INVENTION

Ascorbic acid that can be oxidized easily in a natural environment is usually used as a nutritional additive in the food industry for anti-oxidization. Ascorbic acid has been broadly used in the industry of cosmetics. It has been proven that ascorbic acid has a function of whitening the skin, due to inhibiting the synthesis of tyrosinase, an enzyme enforcing the precipitation of melanin in the dermis. Furthermore, ascorbic acid can provoke the collagen of the derma to grow together and also has a function of tightening the skin.

However, ascorbic acid is very unstable and is degraded easily in a natural environment. Therefore, various derivatives of ascorbic acid, such as ascorbyl magnesium, ascorbyl phosphate, ascorbyl sulphate, ascorbyl glucoside, ascorbyl phosphate, ascorbyl stearate, ascorbyl palmitate, ascorbyl dipalmitate and sodium ascorbyl phosphate, having more stability are provided alternatively. However, these components still have some problems for cosmetics, such as lack of solubility and irritation of the skin due to the pH level thereof.

To resolve the problems, some additives are provided. The additives are then resolved into an oil solvent to stabilize ascorbic acid and derivatives thereof to prolong the shelf life thereof.

Since ascorbic acid and derivatives thereof are classified as water-soluble vitamins, they cannot be dispersed well into the oil and can hardly be absorbed by the skin. Therefore, the effectiveness of whitening the skin provided by ascorbic acid or derivatives thereof can be reduced.

Collagen is degradable, nontoxic, has little antigen, low irritability, high bio-tolerance and bio-absorbability and therefore has been used in the biomaterial industry, medical industry, and chemical industry; and other applications are being found continuously. In addition, collagen is the most occurring material within the derma of a vertebrate, so collagen has been broadly contributed in various types to other cosmetic biomaterials to enhance desired characteristics of a cosmetic. U.S. Pat. No. 4,834,734, No. 4,784,986, No. 3,475,404, No. 3,548,056 and No. 4,140,537 disclose several cosmetics with types of collagen varying in concentrations, molecular weight or solubility, which can contribute to the skin for keeping moisture and removing wrinkles. However, adding collagen in a cosmetic with ascorbic acid still does not resolve the problems aforementioned.

Therefore, it is desirable to provide a cosmetic composition with collagen and a method for stabilizing ascorbic acid or derivatives thereof to enhance the skin to absorb derivatives of ascorbic acid.

### SUMMARY OF THE INVENTION

The objective of the present invention is to provide a cosmetic composition and the method for manufacturing the same to prevent ascorbic acid or derivatives thereof from oxidizing quickly in a natural environment and to enhance the solubility and absorbability of ascorbic acid or derivatives thereof into the skin.

First, a mixed solution with several cosmetic materials is provided. The solute comprises water-soluble collagen with concentrations of about 0.1% to 10% by weight and ascorbic acid or derivatives thereof with concentrations from about 0.1% to 30% by weight. In some embodiments of the present invention, the molecular weight of water-soluble collagen ranges from about 3,000 kilo Dalton (kD) to 300,000 kD. The pH of the rehydrated cosmetic substrate ranges from 4 to 7.

Then, a vacuum dehydration process may be conducted to remove at least 90% water by weight from the mixed solution for forming a cosmetic composition with a porous and three-dimensional structure. In some embodiments of the present invention, the vacuum dehydration process comprises a lyophilization process having a step of water evaporation to make the mixed solution into a solid with a porous and three-dimensional structure.

According to the preferred embodiment, the results of a dissolving test and a stabilization test have determined that after being within the cosmetic composition of the present invention, the solubility of ascorbic acid or derivatives thereof could be increased threefold compared to the commercial products. Moreover, ascorbic acid or derivatives thereof within the rehydrated cosmetic substrate could be preserved from oxidation for at least 12 months without degradation.

### BRIEF DESCRIPTION OF THE DRAWING

The foregoing aspects and many of the attendant advantages of this invention will become more readily appreciated as the same become better understood by reference to the following detailed description, when taken in conjunction with an accompanying drawing, wherein:

FIG. 1 is the flow chart illustrating the processes to manufacture the cosmetic composition, in accordance with a preferred embodiment of the present invention.

### DETAILED DESCRIPTION OF THE PREFERRED EMBODIMENT

The object of the present invention is to provide a cosmetic composition with ascorbic acid or derivatives thereof and enhance derivatives of ascorbic acid to be absorbed by the skin more easily.

The features of the present invention are to utilize the characteristics of the disclosed cosmetic composition to enhance the skin to absorb ascorbic acid or derivatives thereof more effectively, and to increase the shelf life of the same, wherein the water-soluble dehydrated collagen has a porous and three-dimensional structure that can help ascorbic acid or derivatives thereof disperse well in the cosmetic composition, and protect ascorbic acid or derivatives thereof from oxidation. When the cosmetic composition is rehydrated, the solubility of ascorbic acid or derivatives thereof can be increased threefold compared to the commercial products.

In a preferred embodiment of the present invention, the cosmetic composition comprises collagen, ascorbic acid or derivatives thereof, and any additives for a cosmetic product, such as perfume, extender, antioxidant, coloring agent, and colloidal. In some embodiments, the cosmetic composition may be a semi-finished product serving as a cosmetic substrate of an end product for subsequent processes. In other embodiments, the cosmetic composition can be used directly as an end product.

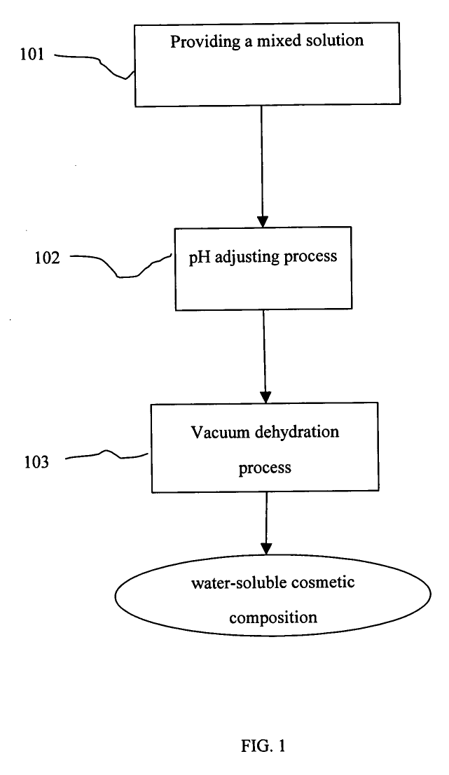

FIG. 1 is the flow chart illustrating the processes to manufacture the cosmetic composition, in accordance with a preferred embodiment of the present invention.

First, referring to step 101, a mixed solution of several cosmetic materials is provided. The solute comprises collagen and other water-soluble or insoluble materials, such as ascorbic acid, derivatives thereof, and any other additives dispersed in the mixed solution.

Collagen is a kind of polypeptide purified from the connective tissue of the vertebrae, wherein water-soluble collagen having lower molecular weight can be well dispersed in water due to the combination of the free water and its hydrogen bond. In the embodiments of the present invention, water-soluble collagen is used to be a dispersing agent with concentrations about 0.1% to 10% by weight and preferably 5%. The molecular weight of the water-soluble collagen may range from about 3,000 kilo Dalton (kD) to 300,000 kD. Since ascorbic acid or directive thereof has varying solubility, the amount of ascorbic acid or the directives thereof may be added depending on what kind of products derived from the ascorbic acid are added in the mixed solution.

In the embodiments of the present invention, the directives of ascorbic acid may comprise levorotary ascorbic acid and its levorotary derivatives and dextrorotary ascorbic acid and its dextrorotary derivatives. Since ascorbic acid is very unstable, it can be degraded easily in a natural environment. Therefore, derivatives of ascorbic acid with more stability, such as ascorbyl magnesium, ascorbyl phosphate, ascorbyl sulphate, ascorbyl glucoside, ascorbyl phosphate, ascorbyl stearate, ascorbyl palmitate, ascorbyl dipalmitate, and sodium ascorbyl phosphate may be used. In a preferred embodiment, the amount of ascorbic acid or the directives thereof may range from about 1% to 30% by weight.

In some embodiments of the present invention, other additives, such as perfume, extender, antioxidant, coloring agent, and colloid may be added in the mixed solution. The concentration of these additives may be added such that it is less than 1% of the mixed solution by weight Referring to step 102, a pH adjusting process may be then conducted to adjust the pH of the mixed solution within the range from 5 to 6.5. Generally, ascorbic acid and derivatives thereof are stable within the range of pH 2 to 4 and the range of 8 to 9.5, which may iritate the skin. In the embodiments of the present invention, ascorbic acid or derivatives thereof within the rehydrated cosmetic substrate could be preserved from oxidation for at least 12 months within the pH range from 4 to 7.

Referring to step 103, a vacuum dehydration process may be conducted with a temperature of less than 60° C. to remove at least 90% water by weight from the mixed solution for forming a cosmetic composition.. A vacuum dehydration process may have a step of water evaporation within an atmosphere less than 1 atm to make the mixed solution into a solid with a porous and three-dimensional structure. The solubility of ascorbic acid or derivatives thereof involved in cosmetic composition may be increased due to the porous and three-dimensional structure. In some embodiments of the present invention, the configuration of the cosmetic composition may be granular powder or a lump with porous and three-dimensional structure. In the preferred embodiment of the present invention, the vacuum dehydration process may be a lyophilization process including several steps as follows. First, the mixed solution is frozen by decreasing the temperature to −15° C. and holding for 1 minute. Then, the operating temperature of 15° C. is maintained for 59 minutes under vacuum of 2 torr. Subsequently, the operating temperature is increased to 35° C. for 2 hours, 45° C. for 2 hours, 55° C. for 1 hour, and 60° C. for 1 hour under vacuum of 2 torr. Then, the vacuum is removed gradually at 60° C. during an interval of 7 hours. In some embodiments of the present invention, the lyophilizated product forms a pile of granular powder. In other embodiments, the lyophilizated product forms a lump.

A lyophilizated product solubility test and a stability test are conducted to compare the cosmetic composition of the present invention with the start materials of the cosmetic composition.

The steps of the solubility test may be as follows: the cosmetic composition is rehydrated with the amount of water equal to that removed from the mixed solution during the lyophilization process to determine the solubility of ascorbic acid or derivatives thereof. Since the solubility varies for different derivatives of ascorbic acid, three repetitions are conducted with different kinds of ascorbic acid derivatives, such as ascorbic acid, magnesium ascorbyl phosphate, and sodium ascorbyl phosphate during the solubility test. The results of the solubility test are described in Table 1:

TABLE 1

| | Magnesium | | Sodium |
|---|---|---|---|
| | ascorbyl | Ascorbic | ascorbyl |
| Solubility | phosphate | acid | phosphate |
| Start materials of the | 0.5 wt % | 3 wt % | 1 wt % |
| cosmetic composition | | | |
| After processing the | 2 wt % | 10 wt % | 5 wt % |
| cosmetic composition | | | |

In accordance with the results, the solubility of the ascorbic acid or derivatives thereof involved in the cosmetic composition can be 3 times greater than the same materials without any processing.

The steps of the stability test are as follows. First, the pH level and activity of ascorbic acid of the rehydrated cosmetic composition are estimated. Then, the rehydrated cosmetic composition is placed in an airtight environment at atmospheric condition. The tests for determining the activity of ascorbic acid within the rehydrated cosmetic are conducted regularly. Since the cosmetic composition has several kinds of recipes, three repetitions of different recipes are conducted during the stability test. The detail description of the repetitions and the results of the stability test are described in Table 2:

TABLE 2

| | Cosmetic composition of | | |
|---|---|---|---|
| | the present embodiment | | |
| | Recipe 1 | Recipe 2 | Recipe 3 |
| Ingredient | Magnesium ascorbyl | Ascorbic acid | Sodium ascorbyl |
| | phosphate | 10 wt % | phosphate |
| | 2 wt % | Water 87 wt % | 5 wt % |
| | Water 95 wt % | Water-soluble | Water 92 wt % |
| | Water-soluble | collagen | Water-soluble |
| | collagen | 3 wt % | collagen |
| | 3 wt % | | 3 wt % |
| PH | 7.23 | 4.3 | 5.6 |
| Activated | >12 months | ≧12 months | >12 months |
| duration | | | |

The results of the stability test illustrate that the pH of the rehydrated cosmetic composition ranges from pH 4 to 7.5, and more preferably from pH 5 to 6.5, approximate to neutrality so as not to irritate the skin. Moreover, the activity of ascorbic acid within the cosmetic composition can be continued for greater than 12 months.

According to the embodiments of the present invention, the water-soluble collagen is facilitated as a disperser of the other cosmetic material. In addition, the porous and three-dimensional structure provided by the lyophilization process can increase the shelf life of derivatives of ascorbic acid. Accordingly, the cosmetic composition can resolve the prior art problems aforementioned.

As is understood by a person skilled in the art, the foregoing preferred embodiments of the present invention are illustrated of the present invention rather than limiting of the present invention. It is intended to cover various modifications and similar arrangements included within the spirit and scope of the appended claims, the scope of which should be accorded the broadest interpretation so as to encompass all such modifications and similar structure.

What is claimed is:

1. A method for forming a water-soluble cosmetic composition comprising:
providing a mixed solution, wherein the mixed solution comprises:
ascorbic acid or a derivative thereof, wherein the concentration of ascorbic acid or a derivative thereof is in the range from about 0.1% to 30% by weight;
a water-soluble collagen, wherein the molecular weight of the water-soluble collagen is between about 3 kD and 300 kD, and the concentration of the water-soluble collagen is between about 0.1% and 10% by weight; and
conducting a vacuum dehydration process to remove a portion of water from the mixed solution.

2. The method for forming the water-soluble cosmetic composition according to claim 1, wherein the concentration of the water-soluble collagen within the mixed solution is about 3% by weight.

3. The method for forming the water-soluble cosmetic composition according to claim 1, wherein the step of providing a mixed solution further comprises adding an additive into the mixed solution.

4. The method for forming the water-soluble cosmetic composition according to claim 1, wherein the vacuum dehydration process may make the water-soluble cosmetic composition shape as a pile of granular powder or shape as a lump.

5. The method for forming the water-soluble cosmetic composition according to claim 4, wherein the water-soluble cosmetic composition has a porous and three-dimensional structure.

6. The method for forming the water-soluble cosmetic according to claim 1, wherein the mixed solution contains 2% ascorbic acid or a derivative thereof by weight.

7. The method for forming the water-soluble cosmetic according to claim 1, wherein ascorbic acid or derivative thereof is selected from a group consisting of levorotary ascorbic acid, derivatives of levorotary ascorbic acid, dextrorotary ascorbic acid, derivatives of dextrorotary ascorbic acid, and any arbitrary combination thereof.

8. The method for forming the water-soluble cosmetic according to claim 8, wherein ascorbic acid or derivative thereof is selected from a group consisting of ascorbic acid, ascorbyl magnesium, ascorbyl phosphate, ascorbyl sulphate, ascorbyl glucoside, ascorbyl phosphate, ascorbyl stearate, ascorbyl palmitate, ascorbyl dipalmitate, and sodium ascorbyl phosphate.

9. The method for forming the water-soluble cosmetic according to claim 1, wherein the step of providing a mixed solution further comprises a pH adjusting process for adjusting the pH of the mixed solution to within the range from 5 to 6.5.

10. The method for forming the water-soluble cosmetic according to claim 1, wherein the vacuum dehydration process removes at least 90% of water by weight from the mixed solution.

11. The method for forming the water-soluble cosmetic according to claim 1, wherein the vacuum dehydration process is selected from a group consisting of a lyophilization process and a vacuum spray process.

12. The method for forming the water-soluble cosmetic according to claim 1, wherein the cosmetic composition rehydrated with the amount of water equal to that removed from the mixed solution by the vacuum dehydration process has a pH in the range from 4 to 8.

13. A water-soluble cosmetic composition made according to the process of claim 1.

14. The water-soluble cosmetic composition according to claim 13, further comprising an additive having a concentration of less than 1% by weight, and the additive is selected from a group consisting of perfume, extender, antioxidant, coloring agent, soluble solid and any arbitrary combination thereof.

15. The water-soluble cosmetic composition according to claim 14, wherein ascorbic acid or derivative thereof is selected from a group consisting of ascorbic acid, ascorbyl magnesium, ascorbyl phosphate, ascorbyl sulphate, ascorbyl glucoside, ascorbyl phosphate, ascorbyl stearate, ascorbyl palmitate, ascorbyl dipalmitate, and sodium ascorbyl phosphate.

16. The water-soluble cosmetic composition according to claim 13, wherein the water-soluble cosmetic composition contains about 10% water by weight.

17. The water-soluble cosmetic composition according to claim 13, wherein the cosmetic composition has a porous and three-dimensional structure.

18. The method for forming the water-soluble cosmetic according to claim 13, wherein the cosmetic composition rehydrated with the amount of water equal to that removed from the mixed solution during the vacuum dehydration process has a pH level between 4 and 8.
